(19) United States
(12) Patent Application Publication
(10) Pub. No.: US 2005/0137466 A1
(43) Pub. Date: Jun. 23, 2005

(54) Clinical curriculum for treatment of compulsive/addictive disorders based on a freedom to change approach

(76) Inventors: Pavel G. Somov, Pittsburgh, PA (US); Marla J. Somova, Pittsburgh, PA (US)

Correspondence Address: QUARLES & BRADY LLP, 411 E. WISCONSIN AVENUE, SUITE 2040, MILWAUKEE, WI, 53202-4497, US

(21) Appl. No.: 11/003,213

(22) Filed: Dec. 3, 2004

Related U.S. Application Data
(60) Provisional application No. 60/527,226, filed on Dec. 5, 2003.

(51) Int. Cl. A61B 5/00

(52) U.S. Cl. 600/300

(57) ABSTRACT

A method for treating compulsive/addictive disorders and particularly drug and alcohol abuse comprises a reason to change, a method to change, and a freedom to change component. The method particularly teaches the client that he or she is free to change by addressing belief structures that may restrict a belief in an ability to change, by training the client that many types of behavior are unthinking and automatic, and by training the client to reinforce the belief that they are free to change through a daily freedom to change routine.

### CROSS-REFERENCE TO RELATED APPLICATION

This application claims the benefit of U.S. provisional patent application Ser. No. 60/527,226, filed on Dec. 5, 2003 and entitled “An Innovative Clinical Curriculum for Substance Use Treatment”, which is hereby incorporated by reference in its entirety.

### STATEMENT REGARDING FEDERALLY SPONSORED RESEARCH OR DEVELOPMENT

Not applicable.

### BACKGROUND OF THE INVENTION

Alcohol and drug abuse in the United States results in damage not only to individuals and their families and friends, but also in a staggering economic cost to society. In a 1992 study published by the National Institute of Health, for example, economic costs from alcohol and drug abuse were estimated at $246 billion. When adjusted for inflation and population growth, these estimates further demonstrate a steady and strong pattern of increase. Furthermore, treatment data suggests that increasing proportions of persons treated for alcohol problems also have drug problems.

The costs attributed to alcohol and drug abuse are derived from a number of factors. These include, for example, motor vehicle accidents and impaired job productivity, costs incurred by the health care system, the crime rate, through crimes committed in order to provide individual support to the user and/or his family, participation in the drug trade, or violence, and job loss, financial destitution, and subsequent reliance on society's safety nets for the user.

Some of these costs are born by the abusers themselves. Such costs include, for example, the loss of legitimate earnings (and household productivity) related to impaired functioning in the labor market, the loss of legitimate earnings related to incarceration, and foregone legitimate earnings when drug abusers pursue income through illegitimate means, including predatory and consensual income-generating crime (e.g., theft, drug trafficking, and prostitution).

These costs, however, are also shifted somewhat to the population as a whole. Lost earnings translate into lost tax revenue (a shift to government), and income from theft accrues to the benefit of abusers—a loss for victims. Other costs, and indeed the majority of costs, moreover, are typically born by the population that does not abuse alcohol and drugs. These increased costs include are accrued in being the victim of drug- and alcohol-related crimes and trauma (e.g., motor vehicle crashes); increased costs of government services, such as criminal justice and highway safety; and increased costs for various social insurance mechanisms, such as private and public health insurance, auto insurance, life insurance, tax payments, pensions, and social welfare insurance.

Because of the great cost of drug and alcohol abuse both to the abusers and to society as a whole, a significant body of work has developed in the area of treatment of compulsive/addictive disorders in general, and the treatment drug and alcohol abuse specifically. While a number of these treatment methods prove very successful for some patients, as can be seen from the data above, significant improvements are necessary to provide treatment to many patients.

Prior art methods of treatment typically rely on developing either a belief in the ability to succeed in a change (self-efficacy; Bandura, 1977) or a “willingness” (Longeran; 1996) to act freely in a patient. Reliance on “universal willingness”, or developing the patient's ability to act freely or willingly rather than automatically, results in the abuser having only a dormant realization or stored philosophy, which is not typically acted on in daily life. These methods, therefore, fail to instill a belief in an ability to change in the patient.

### SUMMARY OF THE INVENTION

The present invention provides a method for the psychological treatment of a compulsive/addictive psychological problem in a patient in accordance with the following equation: Change=Freedom to Change+Reason to Change+Method to Change. The method comprises the steps of providing Choice awareness training to develop awareness of the freedom to change, wherein the choice awareness training is provided prior to the sue prevention training, thereby providing a baseline awareness of the ability to change prior to motivating the patient to change or developing methods to change, providing motivational training to develop a reason to change, and providing use prevention training to develop a method to change. The freedom to change training is provided prior to the motivational training to assure that the patient believes that he or she can change prior to motivating the patient to do so.

In one aspect of the invention, the choice awareness training includes the steps of developing a strategic awareness of freedom to change, developing a tactical awareness of freedom to change, and developing a daily choice awareness plan. The step of developing a strategic awareness of freedom to change comprises training the patient to question their belief structure and to analyze how their belief structure affects their freedom to change, while the step of developing a tactical awareness of freedom to change comprises training the patient to be aware of unconscious conditioned responses and teaching the patient to view their perceived lack of control as an unconscious conditioned response.

In another aspect of the invention, the Choice Awareness Training further comprises the step of providing at least one of an “I” Statement Exercise, an Uncomfortable Chair Exercise, a Choice Step-by-Step Exercise, a Crossing Arms Exercise, a Pointing out Automaticity of Gestures Exercise, a Drawing Choices Exercise, a Signature Exercise, a Choice to Let Go Exercise, a Choice Eye Exercise, a Different Way to Get 4 Exercise, an Arbitrary Choice Exercise, a Who's Doing It Exercise, and an Arbitrary Abstinence Exercise to teach the client of the need to develop an increased baseline of choices.

In another aspect of the invention, the Choice Awareness Training teaches the patient to develop a daily choice awareness practice by discussing conditioning the daily choice awareness practice upon existing conditioned routines and behaviors, examining daily routines including at least one of a daily hygiene, a meal consumption, mobility, posture changes, and a spiritual/religious practices to convert an existing behavior into a choice awareness ritual that is likely to occur on any given day of a patient's life, and selecting a behaviors for the daily choice awareness routine and de-constructing any cognitive-behavioral-affective schemas imbedded in these behaviors to appreciate the opportunities for choice awareness.

In another aspect of the invention, the step of providing Motivational Training to develop a reason to change comprises the steps of providing Motivational Priming, Motivational Enhancement, and Motivational Inoculation, where Motivational Priming comprises providing at least one of a Search-for-Why Self-Help and a Meaning of Life groups, Motivational Enhancement comprises the step of providing Epiphany-Type Motivational Enhancement training, and Motivational Inoculation comprises a Motivation Check Group providing a feedback-based motivational defense group psychotherapy that allows for consolidation of motivation as well as for inoculation against potential motivation-undermining interpersonal encounters.

In yet another aspect of the invention, the Epiphany-Type Motivational Enhancement comprises the developing the understanding of the person's core values to find the dissonance potential by understanding the client's Ideal Self, facilitating the patient's awareness of the dissonance between the ideal and the real self, and deepening the dissonance between the Ideal and the Real Self through at least one of a clinical, experiential, or educational training.

In yet another aspect of the invention, the step of providing a method to change includes providing use prevention training which includes at least one of Slip Prevention, Lapse Prevention, Relapse Prevention and Relapse Termination Training, wherein a slip is defined as having a craving or a desire to engage in a desire to use a substance in question, lapse is defined as acting upon a desire to engage in a target behavior which results in one substance-using episode without a return to the pre-morbid level of substance use, relapse is defined as a repeated episode of using the substance in question following the initial lapse and a return to pre-morbid or nearly pre-morbid level of substance use.

In still another aspect of the invention, the Slip Prevention comprises discussing the concept of Slip and Slip Prevention, identifying internal and external triggers of cravings and desires, and discussing how to avoid the triggers.

In yet another aspect of the invention, the Lapse Prevention training comprises training the patient to recognize a craving, training the patient to diffuse the craving, training the patient leave the scene of the craving, and training the patient to contact a support person to analyze the slip.

In yet still another aspect of the invention, the Relapse Prevention training comprises training the patient to make himself physically comfortable and/or diffusing a craving, training the patient to recognize the lapse and restating one's recovery goals, training the patient to decatastrophize the lapse, training the patient to do at least one of discard the paraphernalia and leave the scene of the lapse, and training the patient to contact the support person to analyze the lapse to prevent another lapse.

In yet another aspect of the invention, the Relapse Termination training comprises the training the client to recognize and amplify the desire to terminate a relapse by at least one of restating his recovery goals and decatastrophizing the consequences of the relapse, training the client to accept the need for external help and the need to rely on external structure, and training the client to access professional help.

In another aspect, the invention provides a method for training a patient with a compulsive/addictive disorder to understand that the patient is free to change. The method comprises the steps of training the patient to developing a strategic awareness of freedom to change, training the patient to developing a tactical awareness of freedom to change, and training the patient to develop a daily choice awareness plan.

The step of training the patient to develop a strategic awareness of freedom to change can comprise training the patient to question their belief structure and to analyze how their belief structure affects their freedom to change. The step of training the patient to develop a tactical awareness of freedom to change can comprise training the patient to be aware of unconscious conditioned responses, and teaching the patient to view their perceived lack of control as an unconscious conditioned response.

In another aspect of the invention, the step of developing a daily choice awareness plan comprises training the client to reinforce choice awareness by consistently making changes in a daily routine, or training the client to condition choice awareness on a high frequency routine.

These and other objects, advantages and aspects of the invention will become apparent from the following description. In the description, reference is made to the accompanying drawings which form a part hereof, and in which there is shown a preferred embodiment of the invention. Such embodiment does not necessarily represent the full scope of the invention and reference is made therefore, to the claims herein for interpreting the scope of the invention.

### BRIEF DESCRIPTION OF THE DRAWINGS

FIG. 1 is a block diagram of a clinical curriculum in accordance with the present invention;

FIG. 2 is a block diagram of the reason to change variable of the curriculum of FIG. 1;

FIG. 3 is a block diagram of the freedom to change variable of the curriculum of FIG. 3; and

FIG. 4 is a block diagram of the method to change variable of the curriculum of FIG. 1.

### DETAILED DESCRIPTION OF THE INVENTION

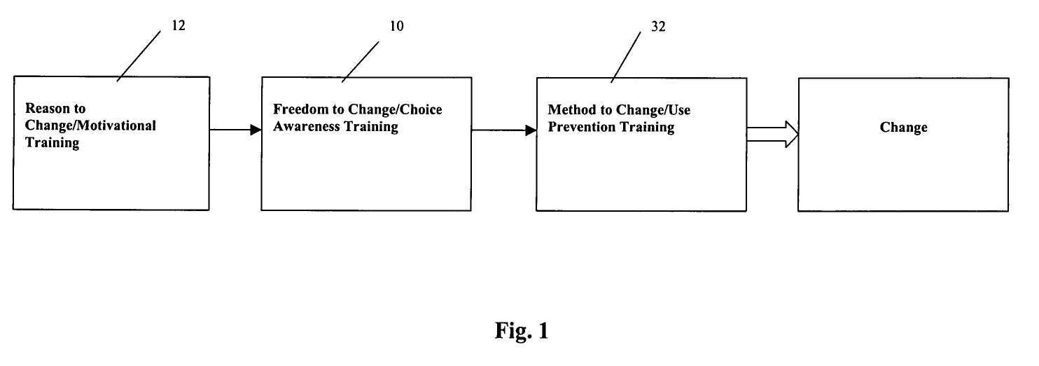

The present invention is a method for treatment of compulsive/addictive disorders which basically includes three steps: Training a patient to understand that they are free to change their behavior, training the patient to understand the reason to change their behavior, and finally training the patient to determine a method for changing their behavior. This training can be stated in the form of a recovery equation clinical curriculum, which, referring now to FIG. 1, can be stated as follows:
Change=Freedom to Change+Reason to Change+Method to Change
This equation will be referred to henceforth as the Change Equation, and is useful in providing an algorithm for patients to guide their progress through treatment and to help patients make sense of post-treatment failures. An important aspect of the change equation is the Freedom to Change component. It is important to train the client to believe that he or she can both initiate and maintain choice awareness, preferably before either motivating the patient to develop a reason to change or developing a method to change, and preferably in the order shown in the equation. In terms of a method for implementation by trainers or facilitators, the Change Equation can be stated in terms of treatment components for recovery, as follows:
Recovery=Choice Awareness Training+Motivational Enhancement+Use Prevention Training

This Recovery Equation is useful for clinicians and can be viewed as an effort to assist the substance use client in developing intrinsic motivation for change that can withstand changes in circumstance, to assist the substance use client in developing strategic and tactical awareness of choice, and to assist the client in developing substance use prevention skills that include craving control, slip prevention, lapse prevention, relapse prevention, relapse termination plans, emotional self-regulation skills and a knowledge-base on the socially-sanctioned, psychologically, physically, financially and legally-safer natural highs, and interpersonal skills. Each of these components will be described more fully below.

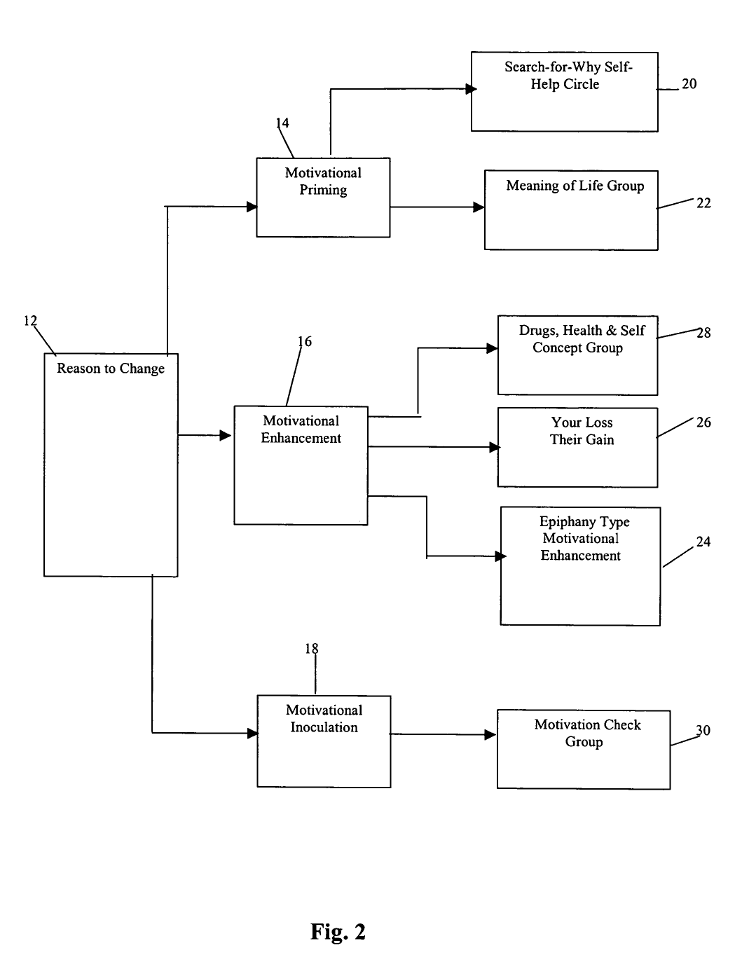

Referring again to FIG. 1, correlating the change and recovery equations, Freedom to Change 10 is developed through Choice Awareness Training in which the patient's freedom-restraining belief structure is explored, questioned and modified to leverage beliefs that support one's freedom-to-change, and cognitive-behavioral-affective automaticity are decreased through a series of experiential exercises and practices intended to deconstruct cognitive-behavioral-affective autopilots and schemas, and through facilitation of a personal daily choice awareness practice. Referring now also to FIG. 2, Reason to Change 12 is developed through Motivational Priming 14, Motivational Enhancement 16, and Motivational Inoculation 18, where the Motivational Priming 14 is provided through Search-for-Why Self-Help 20 and Meaning of Life 22 groups, the Motivational Enhancement 16 is provided particularly through Epiphany-Type Motivational Enhancement 24, and the Motivational Inoculation can be provided through a Motivation Check Group 30. The Method to Change 32 is developed through Use Prevention Training including an array of skill training modalities consisting of Cue-Conditioned Relaxation Training 34, Exposure-Based/Response Prevention Craving Control Training 38, Duality-Based Self-Regulation Training 48, Natural Highs Training 46, and population-specific groups such as Crime and Recovery Group 60.

Training for developing a Freedom to Change 10, a Reason to Change 12, and a Method to Change 32 areas is performed using logical discussion techniques, in dialectic clinical stances which are directed to the following principles:
1) Humanize/Automatize, a dialectic clinical stance that involves Humanization of the abuse problem and Automatization of the Solution to the above problem;
2) Validate/Sublimate, a dialectic clinical stance that is aimed at psycho-dynamically validating and sublimating the personality liabilities of the client and psycho-analytically sublimating these liabilities into socially-acceptable assets;
3) Motivate/Inoculate, a dialectic clinical stance for intrinsically motivating the client to change and inoculating the client from extrinsic changes in circumstance;
4) Catastrophize/Decatastrophize, a dialectic clinical stance that teaches the client to prevent an initial using episode by catastrophizing the negative consequence of the use, and, on the other hand, preventing the further using episode by teaching the client to de-catastrophize the negative consequences of the using episode that had already taken place;
5) De-Automatize/Re-Automatize, a dialectic clinical stance that prompts the client to de-automatize an observed and previously unconscious habitual behavior and prompts the client to re-automatize the de-automatized behavior by an act of conscious choice.

Referring now to FIGS. 2-4, when applied in its entirety in a residential or intensive outpatient setting, it is suggested to arrange the delivery of the various treatment modalities in the order described above in the Change Equation, and as detailed below, wherein the following notations are used to describe frequency of treatment:
“on”—ongoing or semi-ongoing
“pm”—per need/per clinical staff's assessment of need
“opt”—optional (as in “if necessary”)
“V”—check/accountability/summary group
“sh”—self-help groups

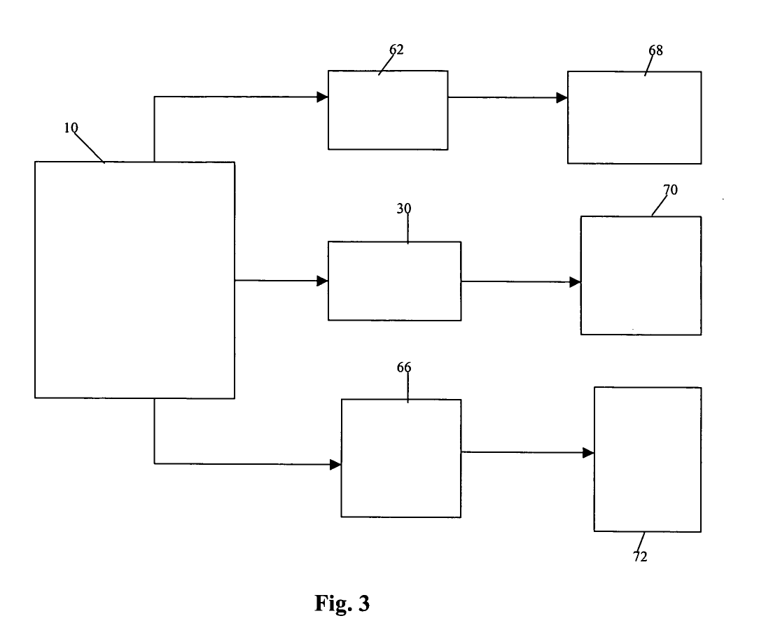

Freedom to Change/Choice Awareness Training. Referring now specifically to FIG. 3, Choice Awareness Training 10 consists of Developing a Strategic Awareness of One's Freedom to Change 62, Developing a Tactical Awareness of One's Freedom to Change 64, and developing a life-long daily Choice Awareness Practice 66. This training comprises the following:
(1) Choice Awareness Training Group
(2-on) Choice Awareness Practice Group
(2-V) Choice Awareness Check Group

Reason to Change/Motivational Enhancement. As described above, the Reason to Change 12 variable includes training in Motivational Priming 14, which explores opportunities for determining meaning; Motivational Enhancement 16, which provides training in enhancing the motivation to change; and Motivation Inoculation 18, which is designed to inoculate against potential motivation undermining interpersonal encounters. The motivation enhancing modalities utilize Festingers (1957) Cognitive Dissonance Theory, while motivation Inoculation utilizes both this theory and Meicherbaum's (1985) Stress Inoculation Training. These can be grouped as follows:

Motivational Priming
(1) Search-for-Why Self-Help Circle 20
(2) Meaning of Life Group 22

Motivational Enhancement
(3) Drugs, Health & Self Concept Group 28
(4) Your Loss—Their Gain Group 26
(pm) Epiphany Type Motivational Enhancement 24

Motivational Inoculation
(5-V) Motivation Check Group 30

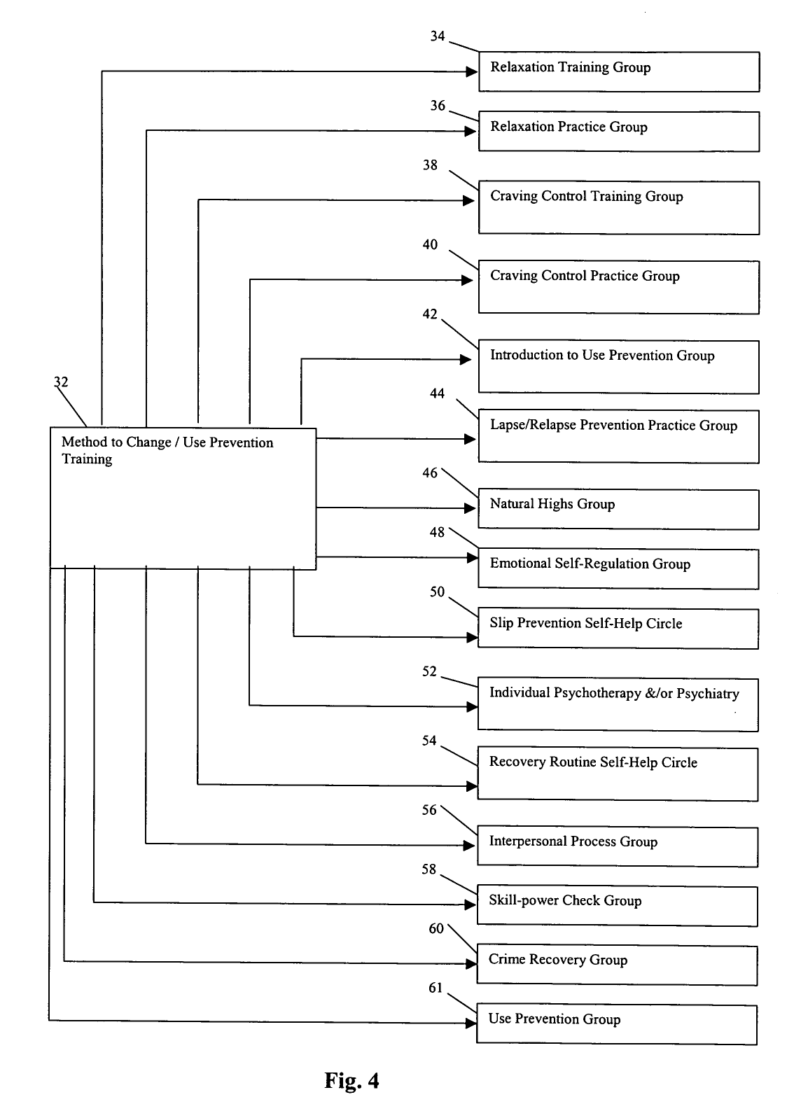

Method to Change/Use Prevention Training. Referring now to FIG. 4, this training is directed toward developing skills in controlling craving, and to preventing both lapses and relapses of use, and can consist of the following:
(1) Relaxation Training Group 34
(2-on) Relaxation Practice Group 36
(3) Craving Control Training Group 38
(4-on) Craving Control Practice Group 40
(5) Introduction to Use Prevention Group 42
(6-on) Lapse/Relapse Prevention Practice Group 44
(7) Natural Highs Group 46
(8) Emotional Self-Regulation Group 48
(9) Interpersonal Process Group 50
(10-V) Skill-power Check Group 52
(pm) Individual Psychotherapy &/or Psychiatry 54
(sh) Slip Prevention Self-Help Circle 56
(sh) Recovery Routine Self-Help Circle 58

Each of the Reason to Change 12, Freedom to Change 10, and Method to Change 32 variables are described more fully under the appropriate headings below.

Change Variable: Freedom to Change

Referring now to FIG. 3, Freedom to Change 10 is induced through the Choice Awareness Training which includes three basic steps: (1) the Development of Strategic Awareness of one's Freedom to Change 62; (2) the Development of Tactical Awareness of one's Freedom to Change 64; and (3) the Development of a Life-long Daily Choice Awareness Practice 66. Choice Awareness Training can also include a Choice Awareness Practice Group, in which choice awareness is practiced, and a Choice Awareness Check Group, which provides feedback to assure that choice awareness continues after training.

Development of Strategic Awareness. Strategic Awareness Training assists the patient in the development of an appreciation of the value of choice awareness and in aiding patients to discard beliefs which stand in the way of their ability to choose, and specifically, in his/her freedom to change the compulsive/addictive behavior presented for treatment. This training consists of a series of guided didactic and experiential exercises for questioning existing belief systems 68 to determine whether they inhibit a belief in freedom of choice. These belief structures can include, for example, a belief that addiction and/or compulsive disorders are part of a disease (“the disease model of addiction”), or various other beliefs which may exist as a function of prior cultural and/or iatrogenic indoctrination.

Development of Tactical Awareness of One's Freedom to Change. Choice Awareness training then proceeds to develop a Tactical (or ongoing) Awareness of One's Freedom to Change. Here, training is directed toward helping patients increase their baseline level of choice awareness through experiential exercises aimed at developing an awareness that one can act freely or willingly at any given moment. Training is directed toward helping the client become aware of the human tendency towards automatic, conditioned, programmed, choice-less, habitual, reflexive, unconscious cognitive-behavioral-affective functioning 70. The training is further directed toward helping the patients appreciate that the automatic or conditioned cognitive-behavioral-affective functioning represents a key asset in human capacity for adaptation, and further that the compulsive/addictive behavior represents a human tendency to find a minimally acceptable coping/problem-solving strategy. The training is further directed toward turning the coping response into an automatic or conditioned cognitive-behavioral-affective coping default until the given coping response is no longer effective.

As described above, the training includes experiential choice awareness exercises that assist the client in developing an appreciation of the need to increase baseline choices. The training can include any or all of the following psycho-educational and psycho-experiential techniques.
(a) “I” Statement Exercise. This exercise includes the steps of asking clients to attempt to “really mean” the pronoun “I” each time they use it in speech in order to raise clients' awareness of whether they use the pronoun “I” by choice or mechanically. When used mechanically, the use is typically mindless, un-free execution of speech autopilots. The training further teaches the clients to incorporate the “I” Statement technique into their daily speech as a choice awareness routine, such that they “really mean” the pronoun “I” when they speak.
(b) Uncomfortable Chair Exercise. This exercise illustrates the interplay between the phenomenon of choice and a delaying of gratification, and involves the following steps: asking clients to assume a mildly uncomfortable sitting posture in a chair; asking clients to become mindful of their discomfort and of their desire to change posture; allowing clients to make a conscious choice of when to change their posture in order to allow them to experience a sense of relief; discussing clients' experience of making a choice in delaying the gratification of the desire to obtain relief from the discomfort of their posture; discussing parallels with the problematic behavior that they have sought treatment for.
(c) Choice Step-by-Step Exercise. This exercise consists of the steps of processing the mindlessness and automaticity of walking, by exploring various choices of pace frequency, stride length, etc., asking the client to consciously choose these various parameters for each step, suggesting that clients actively choose which foot to use for their first step whenever getting up from a chair or from the bed, as a choice awareness routine.
(d) Crossing Arms Exercise. This exercise consists of the asking clients to cross their arms on their chests repeatedly two or three times, processing the habitual, automatic, mindless sequence of the arm crossing; asking clients to cross their arms again but not until they make a conscious choice about which arm goes over which arm, and discussing the phenomenology of making these conscious choices.
(e) Pointing out Automaticity of Gestures Exercise. This exercise consists of the steps of observing clients' in-session behavior and “catching” clients in the process of head nodding, leg shaking, or other gestures and repetitive bodily movements; pointing out the automaticity and the mindlessness of these behaviors; inviting clients to either consciously continue to engage in the highlighted automatic behaviors or to consciously cease to engage in the behavior in question; discussing the phenomenology of taking control over these otherwise involuntary or unconsciously executed processes.
(f) Drawing Choices Exercise. This exercise consists of asking clients to draw several circles one after another; asking the clients to analyze similarities in their drawings; pointing out the automaticity of this behavior (the similarities in diameter, starting points, direction of the drawing); processing the metaphorical meaning of the drawing the same “vicious” cycle or circle; having clients make conscious choice to choose various parameters of the circle-drawing behavior; discussing the difference in the phenomenology of mindless versus mindful circle drawing; suggesting that clients draw one circle consciously each day as a daily choice awareness routine.
(g) Signature Exercise. This exercise consists of having clients sign their name several times in a row; pointing out the mindlessness and automaticity of the signing behavior; asking clients to sign their name mindfully, with awareness; processing how the mindfully executed signature may appear less valid in comparison to a mindlessly executed signature; discussing the pros and cons of mindlessness and automaticity.
(h) The Choice to Let Go Exercise. This exercise consists of instructing the client to clench his/her fist several times in a row and pointing out the apparent mindlessness and automaticity of this behavior; initiating the discussion of various choices available in opening of the fist (the actual choice to open the fist, the timing of the release, the manner of the release (one finger at a time, sequence of the fingers, the whole hand, sudden vs. gradual opening, etc.); having the client clench and consciously release the fist; introducing a metaphor of “letting go” by linking the clenching of the fist to tension and stress and facilitating a discussion of how, at times of stress, people tend to lose their awareness of the opportunities to release the tension and, metaphorically speaking, of the opportunities to let go; asking clients to clench the fist again and providing a metaphor-enhancing narrative (e.g. “You have a choice right now to stay tense or to let go . . . if you decide to let go, you have a choice in how you will let go . . . go ahead and make a choice to either stay tense or to let go . . . if you decided to let go of tension, make a choice on how you will let go of it, all at once, or gradually, by holding on to your tension for a while . . . ”); asking clients to repeat the “letting go” behavior several times, in succession; emphasizing the idea that clients have both a choice to let go or to hold on to their tension, that they have a choice of when to let go, and that they have a choice of how to let go of the tension; pointing out the utility of tension as a physiological cue for letting go; suggesting that clients use the exercise whenever they notice that they become tense or as a daily choice awareness routine.
(i) The Choice Eye Exercise. This exercise consists of instructing the client to imagine that he/she has a Choice Eye, a mind's eye that can see automaticity, schematic behaviors, habits, and mindlessness; instructing the client to let the Choice Eye examine the current moment in search of any behavioral autopilots or schemas, or habits, or mindlessly executed behaviors; instructing the client to choose to continue or cease any given identified cognitive or behavioral or affective autopilot; suggesting that the client perform this exercise for a few minutes each day, letting the Choice Eye take a panaromic view of his or her current moment to detect mindlessness and opportunities for mindfulness.
(j) A Different Way to Get 4 Exercise. This exercise consists of giving the client the following equation on a piece of paper “______+______=4” and asking the patient to solve the equation; if client responds by solving the equation in the following manner, “2+2=4,” he or she is provided with the discussion of how this solution (“two plus two equals four”) represents a common mathematical autopilot; challenging the client to “get 4 through a different solution,” (e.g. “7−3=4,” “569.5−565.5=4,” “8×0.5=4”); stating that if the client chose to, as of this moment, he could come up with a different way to obtain 4, each time, for the rest of his life, even if this is all he did; discussing the phenomenological implication of these previously unappreciated choices; suggesting that the client each day “find a new way to get four;” introducing the metaphor of there being more than one road to Rome or there being more than one way to get 4 or there always being another way to get what one wants.
(k) The Arbitrary Choice Exercise. What would you rather have: red or blue, one or one point three, a glass or a cup? This “offer” is useless since it means nothing. Such offers represent the opportunity for a pure choice. If the client is offered a $20 or $100 bill and asked to choose, the choice is more or less predetermined by the pragmatics. In choice awareness practice, facilitators may offer clients meaningless choices that cannot be guided, i.e. automatized, by considerations of pragmatics or value or ease. Evaluation of two equally meaningless options results in a choice of the purest kind.
(l) The “Who's Doing it?” Exercise. This exercise consists of asking the client to perform a simple motor behavior (such as finger or foot tap or clenching a fist) with a conscious choice preceding each execution of the behavior; following this, the facilitator asks the client to speed up the behavior (such as to increase the pace of finger tapping), at which point the facilitator invites the client to process the issue of agency versus automaticity; more specifically, the facilitator may first ask the client: “clench your fist only after you have made a conscious choice to do so, and do that for a while;” as the facilitator observes the client, a suggestion to speed up the pace of the hand-clenching is tactfully issued; this followed by a discussion of the fact that while clients are able to increase the pace they are not necessarily choosing each contraction; the facilitator then may inquire “Who is doing it?” or facilitators may also engage the client in a conversation while the client continues to clench his or her fist (or other motor behavior such as finger-tapping), and ask: “Who's doing it? Who's clenching the fist (tapping the fingers) while we are talking?”
(m) Arbitrary Abstinence Exercise. This exercise consists of the following steps: facilitator encourages the client to make a choice to abstain from an arbitrarily chosen aspect of his or her life (for example, wearing a particular color of clothing); clients are presented with the rationale for practicing abstinence, but not in application to their particular compulsive/addictive behavior, but as a general concept; furthermore, clients are instructed to make only arbitrary choices about what to quit, to commit to a pre-specified, time-limited abstinence or maintenance (timelines should be initially short and plausible, e.g. a week), and to feel free to break the commitment any time, as long as this arbitrary abstinence is done via a conscious choice.

Life-Long Daily Choice Awareness Practice. Finally, the patient is trained to develop a personalized, Life-Long Daily Choice Awareness Practice 66. This training teaches the patient to reformulate their perceived “loss of control” or “lack of will-power” as an automatic or conditioned cognitive-behavioral-affective functioning, and further helps patients to understand that this cognitive-behavioral-affective conditioning is subject to re-conditioning and re-programming by way of increasing a baseline of choice awareness. The Daily Choice Awareness Practice centers around tying a choice awareness reinforcement to a high frequency or a daily routine 72 of the client, and encourages the client to change the routine on a daily basis to reinforce the belief that choice exists. This Practice is developed using the following tasks:
1. Facilitators explain the need for a regular, daily Choice Awareness Practice as a means to enhancing one's baseline level of choice awareness.
2. Facilitators discuss the paradox of the notion of conditioning the de-conditioning, or automatizing the de-automatization; in particular, clients are explained that the goal is to create a situation in which they will be mindlessly triggered to become mindful and choice-aware.
3. Facilitators present that for a Choice Awareness Practice to become a dependable daily routine it has to be conditioned upon the already existing high-probability and high-frequency conditioned routines and behaviors; alternatively, facilitators explain that the already existent high-probability and/or high-frequency routines can be converted into choice awareness routines.
4. Facilitators examine and explore with clients such high-probability, daily routines as any aspect of daily hygiene (dental hygiene, hand-washing, body-washing, elimination, etc.), or meal consumption, or spiritual/religious practices; with the overall goal of converting an otherwise existing high-probability behavior into a choice awareness ritual; and such high-frequency activities as mobility (walking, standing up, sitting down, etc.), and posture changes (hand clasping, arm crossing, head moving).
5. Facilitators further recommend that the target high-probability and/or high-frequency behavior (upon which a Choice Awareness Practice will be conditioned) be of such nature that it is likely to occur on any given day of a client's life regardless of circumstance and surroundings, with the goal of increasing the overall generalizability of the Choice Awareness Practice to any life circumstance or surrounding.
6. Facilitators help clients select high-probability and/or high-frequency behaviors that are appropriate to be converted into Choice Awareness Routines; and assist clients with de-constructing any cognitive-behavioral-affective schemas imbedded in these behaviors to appreciate the opportunities for choice awareness; and provide client with feedback regarding their emerging Choice Awareness Practice.

When Choice Awareness Training is applied in a residential setting, the theoretical/introductory Choice Awareness Training Group can also be followed by an ongoing experiential Choice Awareness Practice Group, which comprises the following five elements:
1. An ongoing review of the rationale of the choice awareness change variable and of its interplay with other aspects of recovery.
2. Assisting clients with initiating and fine-tuning their daily choice awareness practice.
3. Helping clients process the experiential “fall-out” or insights from their increased choice awareness, and discussion of how clients are applying choice awareness to their various overt and covert cognitive-behavioral-affective autopilots.
4. Providing clients with an in-session structured choice awareness experience both to raise their level of choice awareness and to model ideas for choice awareness applications and practices.
5. Assisting clients with developing a Recovery Autopilot which can be expressed as the following equation, Recovery Autopilot=Daily Recovery Ritual+Weekly Recovery Event(s), the purpose of which is to help clients transition from a structured therapeutic environment to independent self-care, with the Recovery Autopilot serving as a platform for facilitating client's general mental health hygiene and self-care, and consisting of a combination of Relaxation/Meditation, Self-Motivation, Social Support, Choice Awareness activity, Review of Program Materials or some Recovery-related Reading or Study.

Change Variable: Reason to Change

As described above, and referring again to FIG. 2, the Reason to Change 12 module trains the client to evaluate and answer the question “why change”, and comprises the steps of motivational priming 14, motivational enhancement 16, and motivational inoculation 18, each of which are described more fully below.

Motivational Priming. The Reason to Change 12 training begins with Motivational Priming 14 that consists of a Meaning of Life Group 22 and/or a Search-for-Why Self-Help Circle 20. The Search-for-Why Self-Help Circle 20 is introduced immediately after admitting a group/cohort of clients. This self-help circle primes motivation and also leverages such therapeutically-valuable objectives as normalization and validation of one's experience, group cohesion, modeling of self-disclosure, help-seeking, and help-giving. Additionally, the Search-for-Why Self-Help Circle 20 provides leadership opportunities as well as an invaluable opportunity to practice recovery-network building skills. Furthermore, the clients are encouraged to ask “why not?” and “why now?” questions, thus engaging in peer-based motivational inoculation.

The Meaning of Life group 22 is designed to systematically explore various opportunities for meaning. Each group therapy session consists of the following three phases:
1) Lead-in: the facilitator introduces a lead-in item and facilitates the discussion of the issue at hand. Facilitators may use the following 4 types of lead-ins: a quotation, a question, a vignette, an experiential exercise;
2) Personal implications of a given existential value: the facilitator follows-up the discussion with questions that explore personal implications of adhering to a specific existential position;
3) Interplay with substance use: the facilitator facilitates a discussion of the interplay between a particular existential position and substance use.

The Meaning of Life Group 22 therefore offers a systematic exploration of various opportunities for meaning. The Meaning of Life Group 22 is a motivation-enhancing group application of logotherapy that is thematically tailored to the needs of a substance use client. The agenda consists of the following topics and discussion themes:
(1) Introduction to the Scope of Group, Role Induction, & Normalization of Meaninglessness
(2) Meaning of Adversity: What is the meaning of pain & suffering?
(3) Meaning of Me: Who/What am I?
(4) Meaning of Destination: Where am I going?
(5) Meaning of Life: What is the meaning of life? Why am I?
(6) Meaning of Death: What is the meaning of death?
(7) Substance Use as Meaning of Life: What does using do for us?
(8) a) Tentative Answers & Remaining Questions: What do we know?
b) Search Continues: Where do we go from here?

Motivational Enhancement. After the motivational priming process, the training proceeds with Motivational Enhancement 16. Motivational enhancement 16 can include a curriculum designed to enhance a health-based motivation for change, which as shown and is referred to hereafter as the “Drugs, Health & Self-Concept Group” 28. The motivational enhancement further includes training designed to enhance a motivational sentiment of indignation at having been exploited or taken advantage of by legal and illegal drug and alcohol industry, referred to hereafter as the “Your Loss—Their Gain Group” 26.

Motivational enhancement 16 training can also consist, as necessary, of a protocol which is designed to recreate the psychological conditions that precipitate an epiphany-type, sudden-onset pro-change motivational shift (or ETME, epiphany-type motivational enhancement). The epiphany-type training is used as an alternative to the traditional decisional-matrix, gradual-onset motivational enhancement, which is essentially a process of looking at the pros and cons of engaging in a target behavior and pros and cons of not engaging in target behavior. The epiphany-type motivational enhancement follows a different route, aiming at a sudden-onset emotionally (rather than analytically) based motivational enhancement. Here, the training motivates by way of creating and/or re-creating the conditions for an emotional epiphany, and particularly by examining the difference between a person's cognitions (beliefs, abilities, and values) and an action taken, which is also known as cognitive dissonance. This difference can also be described in terms of the client's “Ideal Self” and “Real Self”. This training can include the following tasks and steps:
1. Finding the Dissonance Potential—Find the dissonance (disagreement or incongruity) potential by understanding the client's Ideal Self (who the client wants to be) by developing an understanding of the client's core values.
2. Creating the Dissonance—Create the dissonance between the Ideal Self (who the client wants to be) and the Real Self (who the client really is) by facilitating the client's initial awareness of the dissonance between the ideal and the real self.
3. Deepening the Dissonance—Deepen the dissonance between the Ideal and the Real Self, through clinical, experiential, and educational training.

These tasks can be achieved, in one embodiment of the invention, using the following four session protocols. Each of these protocols consists of both pre-session preparation steps and in-session tasks to be performed by a facilitator or trainer (wherein ME stands for Motivational Enhancement 16):

1. Session 1:

Preparation: The trainer or facilitator reviews what is known about the client on the basis of psychosocial intake data and other history data.

Session: the facilitator establishes rapport with the client and initiates a Decisional Matrix Assessment (both as an appeal to Reason and as a search tool for Dissonance Potentials) which, as described above, is essentially a process of looking at the pros and cons of engaging in a target behavior and pros and cons of not engaging in target behavior. The trainer and client then engage in a Free-flow dialogue that aims at understanding the client's values

2. Session 2:

Preparation: The trainer determines whether she has any intelligent guesses about the client's core values and analyzes possible discrepancies between the clients Ideal and Real Selves to develop working hypotheses about Deepening the Dissonance Potentials in the case.

Session: The facilitator continues to nurture rapport with the client, continue with Decisional Matrix Assessment or Free-flow Dialogue (“value reconnaissance”) until no new personal data are produced, and empathizes if the client has self-discovered and verbalized discrepancies between Ideal and Real Selves.

3. Session 3:

Preparation:
(1) The trainer analyzes results of previous sessions to determine if the client has found a discrepancy between his Ideal and Real Selves. If not, the trainer analyzes steps for helping the client to “stumble” upon the dissonance in the next session. The trainer further analyzes the new personal data learned from the client that sheds light onto who the client is and who they want to be (real/ideal self).
(2) The trainer then states (in writing or mentally) the lead hypothesized Deepening Potentials for a given client.
(3) The trainer states (in writing or mentally) any Dissonance-Deepening statements to be made in the next session and prepares a verbal Deepening Follow-up.
(4) The trainer strategizes and coordinates (logistically) any experiential Dissonance-Deepening strategies that might be employed.

Session: The trainer seeks to Deepen the Dissonance, verbally (if client had self-identified the Dissonance in the previous session) or to Create Dissonance (if client has not yet self-discovered the Dissonance, by stating the lead Deepening Potential).

4. Session 4:

Preparation: The trainer Reviews the experiential Dissonance-Deepening strategy.

Session: The trainer either Deepens the Dissonance, experientially (if used verbal deepening in previous session) or Deepens the Dissonance, verbally, and, then experientially (if no deepening of dissonance was undertaken in previous session).

Motivational Inoculation. Reason to Change 12 training finalizes with Motivational Inoculation 18. Motivational Inoculation 18 consists of a feedback-based motivational defense group psychotherapy modality that allows for consolidation of motivation as well as for inoculation against potential motivation-undermining interpersonal encounters, entitled “Motivation Check Group” 30.

Method to Change

Referring now to FIG. 4, as described above, the Method to Change 32 treatment modalities, particularly when applied in a residential treatment setting, include the following:
A Relaxation Training Group 34
A Relaxation Practice Group 36
A Craving Control Training Group 38
A Craving Control Practice Group 40
An Introduction to Use Prevention Group 42
A Lapse/Relapse Prevention Practice Group 44
A Natural Highs Group 46
A Crime & Recovery Group 48
An Emotional Self-Regulation Group 50
An Interpersonal Process Group 52
A Skill-Power Check Group 54
A Slip-Prevention Self-Help Group 56
A Recovery Autopilot Self-Help Group 58

The “practice groups” are typically intended for use in a residential facility and are typically not used in outpatient or other treatment modalities. While the groups described above are preferably provided, the core training can include the following:
A Craving Control Training Group 38
A Craving Control Practice Group 40
An Use Prevention Group 61
A Lapse/Relapse Prevention Practice Group 44
A Natural Highs Group 46
A Crime & Recovery Group 60
An Emotional Self-Regulation Group 48
   These groups are described in detail below. Whether all of the various modules described above, or the core training set are provided to a patient, the treatment can also include Individual Psychotherapy, Behavioral Medicine and Psychiatry modalities.

Craving Control Training is a two-part psycho-educational/psycho-experiential training process designed to both educate a recovering individual about the nature of cravings and to help a person in recovery to develop a systematic counter-craving response. The training includes the following:
1. Facilitators dispel the myths pertaining to cravings, educate clients about physiology of cravings, and provide clients in substance use treatment with an empirically-concrete definition of a craving, with the craving being defined by the following equation:
   Craving=compulsive thought+(aversive or pleasurable) physiological state
   where the craving is characterized by the following four aspects: a compulsive thought of engaging in a target activity, craving is accompanied by a pleasant or unpleasant physiological reactivity, craving is situation-specific, and craving is time-circumscribed.
2. Facilitators differentiate between stimulus control (“people-places-things” avoidance) strategy and response control (“craving-control”) strategy.
3. Facilitators introduce a combined body/mind craving-control method that is congruent with the physiology of the craving in that the craving control method is behavioral first, and cognitive second, and comprises of the following craving control algorithm:
Recognize the Craving, Relax the Body, Neutralize Craving Thought-Content;
   which can be implemented as follows: “if craving, then relax with the help of cue-conditioned abdominal breathing; if relaxed, then neutralize any craving thought content by reciting a cognitive script”.
4. Facilitators explain the rationale behind the body-first, mind-second sequence of the craving control protocol by discussing how unaddressed physiological reactivity limits cognitive/attentional capacity and, if unaddressed, would render the attempt to neutralize the craving thought-content ineffective.
5. Facilitators present the craving control protocol in a motivation-enhancing metaphor of the craving being a “land-mine consisting of a body-wire and a mind-wire,” with the “body-wire” part of the metaphor referring to physiological aspects of the craving, and the “mind-wire” part of the metaphor referring to the compulsive thought aspect of the craving; and with the process of craving control being metaphorically likened to “diffusing a land-mine by first diffusing a body-wire and then diffusing a mind-wire”. The advantage of the metaphor is that it both guides the execution of the craving control protocol and, at the same time, implicitly reiterates the potential negative consequences of failing to control a craving (failure to control a craving is implicitly linked to a destructive aftermath of failing to diffuse a land-mine), which allows the client the client to internalize the skill of craving control on a motivational level.
6. Facilitators differentiate between less and more optimal craving-control strategies to mobilize clients' motivation for learning and mastering the proposed craving-control strategy, and review and juxtapose the proposed and endorsed craving control procedure versus other craving control strategies such as counter-arguing the craving, distraction, thinking through the craving, aversive conditioning. Facilitators invite clients to examine their experience with these other craving control strategies in the past to allow clients to be in a position of informed consumers.
7. Facilitators provide clients with an opportunity for massed, systematic practice of the proposed craving-control strategy in order to facilitate a sense of mastery, to condition the execution of the craving control response upon the stimulus of the craving, and to desensitize clients to both generic and persons-specific substance use related stimuli.
8. Facilitators, following the educational part of the Craving Control Training, and after the client has been assisted with developing a customized craving control procedure, introduce an opportunity for an exposure/response prevention based practice of the craving control method, with the primary priority being on the massed practice of the method, and the secondary priority being on the desensitization of the client to various generic and/or persona-specific substance use stimuli.

The Craving Control Training Group 38 includes the following 8-session agenda. This training can be applied in a group meeting or applied to individuals. In the individual format, the training is modified to reflect the specifics of one-on-one interaction, but pursues the same overall sequence of theme presentation, which is as follows:

Session 1—Introduction: Craving Control as Part of Lapse/Relapse Prevention
a) introduce the rationale for craving-control as a lapse/relapse prevention strategy
b) explore the role of a craving in participants' lapse history and prime participants for the metaphor: explore the explosive nature of the craving, the undermining nature of a compulsion, the life-blown-into-pieces nature of the post-use consequences
c) introduce the metaphor of a craving as a land-mine, and life after using as a booby-trapped land-mine field (with the help of a craving land-mine exercise).

Session 2—Finding the Balance: Navigation, Not Avoidance
a) educate participants about the difference between stimulus control and response control (craving-control) strategies
b) introduce a clinical metaphor for craving control as a method to “diffuse craving land-mines”
c) introduce the notion of navigation: stimulus control+response control (avoid a craving land-mine+diffuse a craving land-mine)
d) introduce the need for overlearning of the craving-control

Session 3—Understanding the Craving Land Mine
a) dispel craving myths (frequency, intensity, duration, etc.)
b) discuss internal and external craving triggers
c) discuss types of cravings
d) discuss interplay (and lack thereof) between cravings and motivation
e) introduce the idea of self-monitoring as a personal way of dispelling craving myths
f) introduce Craving Diary

Session 4—Understanding the Craving Land Mine (continued)
a) review Craving Diary findings
b) define cravings as consisting of craving thought content and physiological reactivity
c) re-introduce the craving land-mine metaphor translating the two components of a craving into the metaphorical correlates of a “body-wire/body-fuse” and “mind-wire/mind-fuse”

Session 5—Getting Blown Up: Less Optimal Ways to Diffuse the Craving Land-Mine and Better Way to Diffuse the Craving Land-Mine
a) exploring participants' past counter-craving responses
b) discuss counter-craving responses that “blew up” (that failed)
c) introducing the craving control algorithm
d) discuss the logic behind the sequence

Session 6—Diffusing the Body-Wire (Decreasing Bodily Arousal) and Diffusing the Mind-Wire (Decreasing Compulsive Thoughts)
a) discuss the use of relaxation as a means to “diffusing the body-wire/body-fuse”
b) discuss the use of cognitive script as a means to “diffusing the mind-wire/mind-fuse”
c) discuss various types of cognitive scripts
d) facilitate generation of cognitive scripts

Session 7—Customizing the Craving Control Response
a) review, help customize and finalize clients' cognitive scripts
b) review the craving control response

Session 8—Necessity of Exposure as a Craving-Control Practice strategy
a) introduce the notion of exposure/response prevention
b) discuss value of practice of response control strategy
c) discuss value of habituation as a stimulus control bonus
d) demonstrate habituation through repeated exposure
e) address objections to exposure/response prevention as an opportunity for practice of craving control strategy

Craving Control Practice Group. As described above, when applied in a residential setting, the Craving Control Practice Group 40 follows the introductory Craving Control Training 38 and can include an ongoing skill-practice group. The Craving Control Practice Group 38 pursues the following three goals: (1) to help clients over-learn a craving control response to the point of automaticity; (2) to help clients become confident in their ability to tackle any craving they might encounter; and (3) to help clients become proficient in recognizing cravings as early as possible.

These goals are pursued in Craving Control Practice 40 sessions designed to evoke the experience of the craving to trigger the application and the practice of the craving control response. To evoke this response, facilitators use a combination of craving priming and exposure modalities to evoke the craving, using, for example, visual and physical stimuli (slides, pictures, paraphernalia), kinesthetic play-back technique of having a client act out a substance using ritual, and elicitation of reminiscence stories or fantasies pertaining to substance use experience. Craving Control Practice 40 sessions are typically held for groups of 8 individuals, and are typically held daily for thirty minutes or more.

The Craving Control Practice 40 session is physically arranged by the Facilitator to include an Exposure Station which consists of folders or binders with pull-out laminated pictures of substance use related stimuli, organized into various classes of drugs. The slides or pictures represent real life, realistic, non-sterile, non-academic scenes of drinking and/or drugging and/or substance use related socialization. Pictures of paraphernalia are also complimented with pictorial sequences of drug preparation at various stages.

The Facilitator sets up the Exposure Station on one side of the room. Group members are seated in a large circle, with the Facilitator standing near the Exposure Station. The session begins by the person closest to the Facilitator (say, on his left) going to the Exposure Station where he or she exposes him/herself to the pictures to elicit a craving. When the person at the Exposure Station detects a craving, he or she leaves the Exposure Station chair to return to his seat and immediately proceeds with the craving control algorithm. While the first person is engaged in visual self-exposure at the Exposure Station, the Facilitator primes the rest of the group members to experience cravings by a variety of strategies that shall be described separately further below.

If in the course of such priming, a group member who is awaiting his turn to go to the Exposure Station develops a craving, he is expected to initiate a craving control algorithm on his own initiative, without prompting by the Facilitator. As noted above, when a person at the Exposure Station develops the craving, he immediately returns to his seat in the group to perform a craving control algorithm; this act of leaving experientially hard-wires the strategy of combining craving control with at least temporary leaving of the scene of the craving, whenever possible. This avoidance maneuver, however, is not available to the person who got triggered by the Facilitator's priming. As soon as the Exposure Seat is vacated, the next person in line proceeds to the Exposure Station, unless that person is a person who was triggered by the Facilitator's priming and is currently in the process of diffusing his cravings.

It should be noted that whenever a person is in the process of diffusing a craving, he is asked to remain unresponsive to any social engagement. This is done to reiterate the importance of craving control before anything else. Occasionally, the facilitator may intentionally or unintentionally reach out to a person who is in the process of diffusing a craving. In such case, if the person does not acknowledge the social engagement, he is given positive feedback afterward for doing “first things first”.

When group members are finished diffusing their cravings, they record their experience in the Craving Control Success Record; this is done after each registered craving that transpired. As the session unfolds, the facilitator processes the experience with those group members that have either completed a given round of exposure or had not yet embarked on one; in doing so, the Facilitator oscillates between processing, giving feedback, encouraging documentation, and continuously priming additional cravings for either those he is conversing with directly or those who just returned to their seats from the Exposure Station. The group rotates in this manner for the duration of the session, with the format of the session being akin to a psychological circuit training where the person moves from tasks of exposure to craving control to documentation to analysis and processing to exposure.

Facilitators address any arising objections to clients' participation in the Craving Control Practice Group 40 by being prepared to discuss the following common sentiments:
a. “This will make me have a craving!” This objection is addressed by going over one of the metaphors for the rationale for use of exposure such as craving control being an opportunity to “shadow box”. Hesitation and concern are validated as normal. Clients are explicitly discouraged from any attempts at self-exposure between sessions, at least initially, and are assured that if, in the course of a session they develop a strong craving that they feel they cannot diffuse, they will be assisted with extinguishing it in the course of the session and after the session, if necessary.
b. “This will not make me have a craving!” This objection is easily tackled by handing the client a picture of his drug of choice. In preparing the pictures for exposure, it is recommended that facilitators use high resolution color pictures of drugs and/or drug related paraphernalia, with the size of the image approximating the actual objects. An example of an effective picture for crack cocaine is an actual-size picture of a person's hand, palm up, with a nice piece of “rock”. A cut-out of a hand, palm-up, with a piece of “rock,” would be an effective counter-argument to the notion that a “picture will not give me a craving”.
c. “I should not have a craving!” This type of objection is a function of confusing the fact of the craving with one's state of motivation. Clients are reminded that the fact of having a craving is but a reflection of the fact that they had conditioned themselves to associate certain stimuli with certain substances. Consequently, having a craving is not a motivational crisis but a mere reflection of their past history. In regard to purposeful elicitation of cravings, clients are encouraged to evaluate the pros and cons of engaging or not engaging in this form of skill training. Their self-imposed ban on cravings is validated for its long-term motivational value and exposure-based craving control practice is presented as a challenging sacrifice that can be only justified by a strong desire to quit using.

Facilitators assist clients with balancing the training need to have craving control success precedents with progressively more intense cravings and the training need to condition the execution of the craving control response at the first signs of a craving.

Facilitators also present the rationale behind the Craving Control Success Record which is designed to enhance client's sense of mastery, and consists of a form that allows the client to document the fact of the craving, its intensity, its duration, and whether the client has successfully controlled it or not.

Use Prevention. Use Prevention 61 refers, particularly in application to a substance use treatment setting, to a combination of Slip Prevention (SP), Lapse Prevention (LP), Relapse Prevention (RP) and Relapse Termination Plans (RT). Total Use Prevention is expressed as the following equation:
Use Prevention Skill-Power=SP Plan+LP Plan+RP Plan+RT Plan.
Where Prevention Skill-Power Training, if implemented in a residential setting, consists of a Prevention Skill-Power Training Group, a Slip Prevention Self-Help Group, and Lapse/Relapse Prevention Practice Group.

As part of the Use Prevention training, facilitators use a so-called Banana Peel metaphor to make distinctions between Slip, Lapse, Relapse and the tasks of Slip Prevention, Lapse Prevention, Relapse Prevention, and Relapse Termination. Slip is defined as having a craving or a desire to engage in a target behavior, a desire to use a substance in question. Lapse is defined as acting upon a desire to engage in a target behavior which results in one substance-using episode without a return to the pre-morbid level of substance use. Relapse is defined as a repeated episode of using the substance in question, following the initial lapse, and a return to pre-morbid or nearly pre-morbid level of substance use.

Slip Prevention is defined as prevention of unnecessary cravings or desires to engage in a target behavior by avoiding the avoidable triggers of such cravings and desires. Slip Prevention consists of the following steps: introducing the notion of Slip and Slip Prevention, assistance to clients with identifying internal and external triggers of their cravings and desires, and either discussing individually how to avoid the avoidable triggers, or, if in residential setting, providing clients with an opportunity to participate in a Slip Prevention Self-Help Group.

Lapse Prevention is defined as prevention of actual engagement in a behavior in question once the person has, in fact, encountered an internal or external trigger, and as a result has developed a desire or a craving to engage in the behavior in question. Lapse Prevention can consist of the following steps: recognizing the presence of a craving, diffusing the craving; leaving the scene of the craving if possible, contacting support person, and analyzing the slip to prevent the next slip.

Relapse Prevention is defined as preventing a return to the pre-morbid or nearly pre-morbid level of engagement in the target behavior once a lapse has already occurred. Relapse Prevention consists of the following steps: making oneself physically comfortable and/or diffusing a craving (if applicable); recognizing the fact of the lapse and restating one's recovery goals; decatastrophizing the fact of the consequences of the lapse; discarding the paraphernalia and/or leaving the scene of the lapse; contacting the support person and analyzing the lapse to prevent the next lapse.

Relapse Termination is defined as a plan for terminating a protracted relapse. Relapse Termination consists of the following steps: recognizing and amplifying the desire to terminate a relapse by restating one's recovery goals and/or decatastrophizing the consequences of the relapse; accepting the need for external help (as one's internal resources have been by now exhausted) and the need to rely on external structure (such as a residential program) temporarily while one is strong enough to rely on his recuperated internal resources; and accessing professional help immediately. As part of Relapse Termination training, clients are instructed with specific ways of dealing with various logistical and emotional obstacles to accessing help.

Lapse Prevention Practice Group. The Lapse/Relapse Prevention Practice Group 44 pursues the following goals:
1. Providing an opportunity to practice prevention plans through a variety of role-plays and hypothetical scenarios.
2. Allowing clinicians to observe and provide feedback to clients about their prevention skills.
3. Allowing the clients to enhance their sense of use prevention self-efficacy.

The Lapse/Relapse Prevention Practice Group uses the following modalities to pursue these goals:
1. Critique/feedback of role-plays by clinicians, role-play participants, and fellow observers.
2. Actual behavioral scripted and non-scripted role-plays. When engaging in behavioral role-plays, clients are instructed to enact their prevention plans in real time (e.g. not merely announcing that in a given situation they would attempt craving control, but instead engaging in craving control). When directing behavioral role-plays facilitators aim to allow clients to re-play the role-plays to the point of satisfying resolution of a given hypothetical difficulty to facilitate a sense of mastery and success in skill acquisition. While setting up a role-play, facilitators designate a “voice of the craving” role for a willing role-play participant to simulate the internal dialogue (craving-related thought-content, etc.) for the protagonist of the role-play. While guiding a role-play facilitators are encouraged to suggest unexpected obstacles to various points on clients' prevention plans (such as not being able to contact the support person) to allow for a role-play experience that is generalizable to the real world conditions.
3. Discussions of hypothetical scenarios. Following the role-plays the facilitators process personal lessons from the role-play for all parties involved.
4. Graphic reproduction of clients' plans and audio-taping of their own prevention memos that they can listen to as part of their Recovery Autopilots and/or to play-back on demand when necessary post-treatment. As part of the Lapse/Relapse Prevention Practice Group, group participants are instructed to audio-tape their own Prevention Memos where, in the case of audio cassette as a medium, one side could be devoted to a self-directed Motivational Enhancement Appeal and the other side could be devoted to the recording of one's Slip, Lapse, Relapse Prevention Plans, and Relapse Termination Plan; Clients are encouraged to make their recordings available for either peer or staff feedback.

Natural Highs Training. The Natural Highs 46 training represents a systematic lifestyle re-balancing tool that provides substance use clients with an intuitive taxonomy of feeling states with a corresponding matrix of healthy or healthier alternatives, by presenting information on socially-sanctioned, psychologically, physiologically, and financially safer alternatives to “feeling good”.

The goals of the Natural Highs training are:
a) to validate clients' desire to “get high” or to “slow down” or to “feel normal” or “to experiment” as normal human desires, and, therefore, to provide a non-pathologizing view of clients' substance use as a less optimal form of coping or emotional self-regulation;
b) to introduce alternative, natural, socially acceptable, psychologically, financially and legally safer mood-altering strategies, as a more optimal means to an otherwise legitimate goal of “feeling good”;
c) to reiterate the utmost importance of having a “feel good” know-how as a means of slip prevention through establishment of an emotionally fulfilling and balanced lifestyle;
d) to expand clients' awareness of various natural, socially acceptable, physically, financially and legally safer alternatives to specific drug classes;
e) to assist clients in learning to appreciate the benefits of behavioral activation as well as of socially-sanctioned and legal psycho-physiological activation;
f) to assist clients in developing a leisure menu of what they know they like to do and of the activities they would like to experientially sample; and
g) to facilitate clients' integration of the behavioral activation emotional self-regulation strategies into their daily routines while they are in a therapeutic program, in a proactive manner.

The Natural Highs Group can consist of the following format:
a) Introduce a given “natural high” strategy, then describe the activity and/or legal substance associated with a given “natural high” mood-enhancing strategy.
b) Discuss the expected physiological effects of a given natural/legal mood-enhancing activity or substance.
c) Draw parallels between the natural/legal strategy in question and illegal drug of a class that produces similar effects.
d) Discuss the group's experience with a given strategy and elicit comparisons between the natural/legal strategy in question and the corresponding illegal strategy.
e) Discuss relative cons and pros of a given natural/legal strategy.
f) Discuss costs and the logistics/pragmatics of a given natural/legal strategy.
g) Elicit statements of intention regarding future involvement or at least experiential sampling of a given “natural high” mood-enhancing strategy.

Crime and Recovery Group. The Crime and Recovery Group 60 provides a systematic discussion of the interplay between crime and substance use recovery. This group is particularly applicable in a correctional treatment setting, and consists of the following goals and methods:

The Crime and Recovery Group articulates the following goals:
a) To educate correctional substance use treatment clients about the psychological interplay between crime and substance use, with the purpose of assuring their recovery.
b) To assist correctional substance use treatment in determining what role crime plays in their life.
c) To assist correctional substance use treatment in developing a plan for meeting their psychological and financial needs through non-criminogenic, socially-sanctioned avenues.

The Crime and Recovery Group offers the following conceptualization of the interplay between crime and substance use:
1) Instrumental (a person engages in crime as a means to obtain property/finances to buy drugs).
2) A form of a high (crime is committed explicitly for the purpose of self-stimulation, “rush” feeling from engaging in a risk-taking behavior).
3) A form of ego-boosting (crime is committed explicitly for the purpose of a sense of “getting over,” control, superiority over others, self-aggrandizement).
4) A form of belonging (crime is committed for the purpose of gaining entrance to or maintaining membership in a given social strata, e.g. gang, the “neighborhood,” etc.).
5) Or as a combination of the above.

The Emotional Self-Regulation Training. The Emotional Self-Regulation Training 48 consists of both traditional (not meta-cognitive) Emotional Self-Regulation and Duality-Based (Meta-Cognitive) Emotional Self-Regulation training modalities. Each of these are described more fully below.

The overall Self-Regulation training can be expressed as the following equation:
Self-Regulation=(1) (Subject Distance+Object Nonchalance)+(2) (Object Modification or Object Negation),
Here, the first component refers to Duality-Based (Meta-Cognitive) Emotional Self-Regulation training that allows a client to learn to access a state of mind in which the person feels both separated from his/her internal objects (such as thoughts, feelings, sensations) and also appreciates their relative or absolute meaninglessness. The second component consists of traditional Emotional Self-Regulation training which comes into play if the original object (thought, feeling, sensation) is either novel or recurrent and minor in its emotional intensity, in which case, the person is taught to modify the object (through the classic cognitive modification route) or may negate/discard the object altogether by changing one's thought content through behavioral activation or cognitive distraction.

As part of the Duality-Based Emotional Self-Regulation training, facilitators teach clients to separate one's Self from one's experience, i.e. to cognitively separate the Regulator from the Regulated. A client learns about the phenomenological distinction between Self as a Subject (or the Regulator) and Self as an Object (or the Regulated). This distinction is conveyed through a practice of meditative steps which help the client learn to access this sense of duality, this phenomenological separation or estrangement from one's thoughts, feelings, or sensations. This may be accomplished through theoretical discussions, various metaphors, am-ness meditations, and experiences designed to leverage a state of meta-cognition.

As part of the Duality-Based Emotional Self-Regulation training, facilitators teach clients that all experience is arbitrary (i.e. Chosen) and meaningless. The client is taught to appreciate that all objects, internal and external, are relatively or absolutely meaningless, with their meaning being fundamentally a matter of client's choice of interpretation. This philosophical nonchalance helps the client be less reactive and less attached to his/her objects. and is accomplished through discussions of the distinction between the objective and the subjective and through a Philosophical Nonchalance exercise that helps clients understand that all objects, internal or external, contain no meaning and that whatever meaning they might evoke is arbitrary in nature and represents a subjective, interpretive choice by the perceiver.

As part of the Duality-Based Emotional Self-Regulation training, facilitators also help client to identify a reasonable emotional baseline and to condition this meta-cognitive self-regulation to any significant ego-dystonic deviation from the baseline. The client is instructed to only use duality-based self-regulation when there is a substantial ego-dystonic deviation from the emotional baseline, while allowing normal cognitive-behavioral-affective homeostatic processes to manage minor deviations from the emotional baseline.

As part of the traditional Emotional Self-Regulation training, clients are taught to acquire, practice, and apply traditional (non-meta-cognitive) modification of thoughts as a means to changing one's feelings and/or behavioral activation. Clients are taught a standard repertoire of emotional self-regulation skills inclusive of cognitive modification of thought distortions and behavioral activation. These skills are used for back-up or for trying to understand/process new psychological Objects.

In sum, the total Self-Regulation training consists of: leveraging motivation for self-regulation; introduction of distinction between the Regulator and the Regulated; demonstration of the distinction between the Regulator and the Regulated; introduction of notion of emotional baseline; personalization of emotional baseline; conditioning of the Regulator's emergence on deviation from baseline through in-session exposure and/or system of reminders; introduction of the notion that all experience is arbitrary and subject to regulation; internalization of experiential relativity via philosophical nonchalance meditation; introduction of selected non-meta-cognitive regulatory strategies; packaging self-regulation into a two-step process of, first, accessing the Regulator (via the separation of the Regulator from the Regulated) and, second, regulating the experience with the help of traditional non-meta-cognitive techniques until a return to emotional baseline had been accomplished); emotion-specific discussions (guilt, shame, anger, boredom, etc.); making an inventory of one's emotional autopilots and re-programming thereof.

Summary

The present method, therefore, provides a clinical curriculum for treating compulsive/addictive disorders which relies on both a why component (a Reason to Change), a how component (a Method to Change). Furthermore, the present method includes training the patient to understand that they are free to change, to help to ensure actual change or recovery results.

The development of awareness of and practice of freedom of choice on the part of the client is provided by training the client to analyze belief structures that are impediments to free choice, training the client to recognize that many actions are automated but can be changed by free choice, and teaching the client to develop an awareness plan which daily reinforces the understanding that the client is free to choose his or her own actions.

The present method further provides improved methods of providing a reason to change, which rely on motivational training including meaning of life and epiphany training. Additionally, the method provides improved use prevention training by clearly defining various aspects of use (slip, lapse, relapse) and teaching methods for dealing with all of these various types. The method also provides training in emotional self-regulation to aid the client in controlling cravings as well as dealing with slips, lapses, and relapses.

It should be understood that the methods described above are only exemplary and do not limit the scope of the invention, and that various modifications could be made by those skilled in the art that would fall under the scope of the invention. To apprise the public of the scope of this invention, the following claims are made:

1. A method for the psychological treatment of a compulsive/addictive psychological problem, comprising the following steps:
providing choice awareness training to develop awareness of the freedom to change;
providing motivational training to develop a reason to change; and
providing use prevention training to develop a method to change;
wherein choice awareness training is provided to develop a belief in an ability to initiate and maintain choice awareness prior to the motivational training.

2. The method of claim 1, wherein choice awareness training includes the steps of developing a strategic awareness of freedom to change, developing a tactical awareness of freedom to change, and developing a daily choice awareness plan.

3. The method of claim 2, wherein the step of developing a strategic awareness of freedom to change comprises the step of training the patient to question their belief structure and to analyze how their belief structure affects their freedom to change.

4. The method of claim 2, wherein the step of developing a tactical awareness of freedom to change comprises the steps of:
a) training the patient to be aware of unconscious conditioned responses;
b) teaching the patient to view their perceived lack of control as an unconscious conditioned response; and
c) training the patient to develop a moment-by-moment awareness of a freedom to change.

5. The method of claim 2, wherein the step of developing a daily choice awareness practice further comprises the step of providing at least one of an “I” Statement Exercise, an Uncomfortable Chair Exercise, a Choice Step-by-Step Exercise, a Crossing Arms Exercise, a Pointing out Automaticity of Gestures Exercise, a Drawing Choices Exercise, a Signature Exercise, a Choice to Let Go Exercise, a Choice Eye Exercise, a Different Way to Get 4 Exercise, an Arbitrary Choice Exercise, a Who's Doing It Exercise, and an Arbitrary Abstinence Exercise to teach the client of the need to develop an increased baseline of choices.

6. The method of claim 2, wherein the step of teaching the patient to develop the daily choice awareness practice using the following steps:
discussing conditioning the daily choice awareness practice upon existing conditioned routines and behaviors;
examining daily routines including at least one of a daily hygiene, a meal consumption, a mobility, a posture change, and a spiritual/religious practice to convert an existing behavior into a choice awareness ritual likely to occur on any given day; and
selecting a behavior for the daily choice awareness routine and de-constructing any cognitive-behavioral-affective schemas imbedded in these behaviors to appreciate the opportunities for choice awareness.

7. The method as recited in claim 1, wherein the step of providing Motivational Training to develop a reason to change comprises the steps of providing Motivational Priming, Motivational Enhancement, and Motivational Inoculation.

8. The method as recited in claim 7, wherein the step of providing Motivational Priming comprises providing at least one of a Search-for-Why Self-Help and a Meaning of Life groups.

9. The method as recited in claim 7, wherein the step of providing Motivational Enhancement comprises the step of providing Epiphany-Type Motivational Enhancement training, and wherein the Epiphany-Type Motivational Enhancement training comprises the following steps:
a) developing the understanding of the person's core values to find the dissonance potential by understanding the client's Ideal Self;
b) facilitating the patient's awareness of the dissonance between the ideal and the real self; and
c) deepening the dissonance between the Ideal and the Real Self through at least one of a clinical, experiential, or educational training.

10. The method as recited in claim 7, wherein the step of providing Motivational Inoculation comprises a Motivation Check Group providing a feedback-based motivational defense group psychotherapy that allows for consolidation of motivation as well as for inoculation against potential motivation-undermining interpersonal encounters.

11. The method as recited in claim 1, wherein the step of providing a method to change comprises the step of providing use prevention training, the use prevention training comprising at least one of a Slip Prevention, a Lapse Prevention, a Relapse Prevention and a Relapse Termination Training.

12. The method as recited in claim 11, wherein a slip is defined as having a craving or a desire to engage in a desire to use a substance in question, lapse is defined as acting upon a desire to engage in a target behavior which results in one substance-using episode without a return to the pre-morbid level of substance use, relapse is defined as a repeated episode of using the substance in question following the initial lapse and a return to pre-morbid or nearly pre-morbid level of substance use.

13. The method as recited in claim 11, wherein Slip Prevention comprises the following steps:
discussing the concept of Slip and Slip Prevention with the patient;
identifying internal and external triggers of cravings and desires in the patient; and
discussing how to avoid the triggers with the patient.

14. The method as defined in claim 11, wherein Lapse Prevention training comprises the steps of:
training the patient to recognize a craving;
training the patient to diffuse the craving;
training the patient to leave the scene of the craving; and
training the patient to contact a support person to analyze the slip.

15. The method as defined in claim 12, wherein Relapse Prevention training comprises the following steps:
training the patient to make himself physically comfortable and/or to diffuse a craving;
training the patient to recognize the lapse and to restate the patient's recovery goals;
training the patient to decatastrophize the lapse;
training the patient to perform at least one of discarding the paraphernalia and leaving the scene of the lapse; and
training the patient to contact the support person to analyze the lapse.

16. The method as defined in claim 11, wherein Relapse Termination training comprises the steps of:
training the client to recognize and amplify the desire to terminate a relapse by at least one of restating his recovery goals and decatastrophizing the consequences of the relapse; and
training the client to accept the need for external help and the need to rely on external structure; and new line training the client to access professional help.

17. The method of claim 1, wherein the Method to Change treatment modalities comprise at least one of a Relaxation Training Group, Relaxation Practice Group, Craving Control Training Group, Craving Control Practice Group, Introduction to Use Prevention Group, Lapse/Relapse Prevention Practice Group, Natural Highs Group, Crime & Recovery Group, Emotional Self-Regulation Group, Interpersonal Process Group, Skill-Power Check Group, Slip-Prevention Self-Help Group, Recovery Autopilot Self-Help Group.

18. The method of claim 17, wherein the Emotional Self-Regulation Training consists of both traditional Emotional Self-Regulation and Duality-Based Meta-Cognitive Emotional Self-Regulation training as expressed in the following equation:
Self-Regulation=(1) (Subject Distance+Object Nonchalance)+(2) (Object Modification or Object Negation),
wherein the first component refers to Duality-Based (Meta-Cognitive) Emotional Self-Regulation training to teach the client to access a state of mind in which the client feels separated from his/her internal objects and appreciates their relative or absolute meaninglessness and the second component consists of traditional Emotional Self-Regulation training in which the object is implemented when either novel or recurrent and minor in its emotional intensity, in which case, the person is taught to perform at least one of modifying the object or negating the object by changing the client's thought content through behavioral activation or cognitive distraction.

19. The method as recited in claim 18, further comprising the step of teaching a client to separate the client's self from the client's experience through at least one of meditative steps, theoretical discussions, metaphors, meditations, and experiences designed to produce a state of meta-cognition.

20. The method as recited in claim 18, further comprising the step of teaching the client that all experience and all objects are arbitrary and meaningless to teach the client to be less reactive and less attached to objects through at least one of a discussion of the distinction between the objective and the subjective, and a philosophical nonchalance exercise.

21. The method as recited in claim 18, further comprising the step of training clients to identify a reasonable emotional baseline and to condition a meta-cognitive self-regulation to any significant ego-dystonic deviation from the baseline.

22. The method as recited in claim 21, further comprising the step of teaching the client to acquire, practice, and apply traditional non-meta-cognitive modification of thoughts to change the client's feelings and/or behavioral activation.

23. A method for training a patient to understand that the patient is free to change, the method comprising the following steps:
training the patient to develop a strategic awareness of freedom to change;
training the patient to develop a tactical awareness of freedom to change; and
training the patient to develop a daily choice awareness plan.

24. The method as recited in claim 23, wherein the step of training the patient to develop a strategic awareness of freedom to change comprises the step of training the patient to question their belief structure and to analyze how their belief structure affects their freedom to change.

25. The method as recited in claim 23, wherein the step of training the patient to develop a tactical awareness of freedom to change comprises the steps of
a) training the patient to be aware of unconscious conditioned responses; and
b) teaching the patient to view their perceived lack of control as an unconscious conditioned response; and
c) training the patient to develop a moment-by-moment awareness of freedom to change.

26. The method as recited in claim 23, wherein the step of developing a daily choice awareness plan comprises training the client to reinforce choice awareness by consistently making changes in a daily routine.

27. The method as recited in claim 23, wherein the step of developing a daily choice awareness plan comprises training the client to condition choice awareness on a high frequency routine.

28. A method for training a patient to understand that the patient is free to change, the method comprising the following steps:
a) training the patient to question their believe structure and to analyze how their belief structure affects their freedom to change;
b) training the patient to develop a moment-by-moment awareness of a freedom to change; and
c) training the patient to develop a choice awareness plan;
wherein the choice awareness plan is based on a high frequency activity which the patient consistently changes to reinforce a belief in the freedom to change.

29. The method as recited in claim 28, wherein high frequency activity comprises at least one of a daily hygiene routine, a meal consumption routine, a spiritual routine, a mobility routine, or a posture change routine.

30. The method as recited in claim 28, wherein the step of training the patient to develop a moment-by-moment awareness of a freedom to change comprises the step of prompting the patient to perform at least one experiential choice awareness exercise.

31. The method as recited in claim 28, wherein the step of training the patient to develop a moment-by-moment awareness of a freedom to change comprises training the patient to be aware of the human tendency toward an automatic conditioned response.
